(19) United States
(12) Patent Application Publication
(10) Pub. No.: US 2008/0243204 A1
(43) Pub. Date: Oct. 2, 2008

(54) VARIATIONAL PARAMETER NEUROSTIMULATION PARADIGM FOR TREATMENT OF NEUROLOGIC DISEASE

(75) Inventors: Basim Uthman, Gainesville, FL (US); Panagote Pardalos, Gainesville, FL (US); Michael Andrew Bewernitz, Gainesville, FL (US); Chang-Chia Liu, Gainesville, FL (US); Stanislav Busygin, Bala Cynwyd, PA (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Gainesville, FL (US)

(21) Appl. No.: 12/056,696

(22) Filed: Mar. 27, 2008

Related U.S. Application Data
(60) Provisional application No. 60/908,591, filed on Mar. 28, 2007.

(51) Int. Cl. A61N 1/36

(52) U.S. Cl. 607/45

(57) ABSTRACT

The present invention concerns a neural stimulation device, and methods for its use, in which one or more stimulation parameters can be automatically and randomly adjusted such that any particular combination of stimulation parameters is not repeated for a given duration of time, thereby limiting habituation to the neural stimulus.

### CROSS-REFERENCE TO RELATED APPLICATION

The present application claims the benefit of U.S. Provisional Application Ser. No. 60/908,591, filed Mar. 28, 2007, which is hereby incorporated by reference herein in its entirety.

### BACKGROUND OF THE INVENTION

Many neurostimulation devices deliver electrical stimulation to the central nervous system (CNS) at periodic time intervals, such as the vagus nerve stimulator used in VNS therapy. While under the influence of the periodic stimulation paradigm, individuals receiving medical treatment by the neurostimulation device may experience an eventual relapse of the symptoms of their respective disorder or disorders of the CNS. The relapse of symptoms of the disorder could be due to the disorder adapting to sufficient number of repetitions or a sufficient repetition duration of identical stimulations equally spaced in time. One example of this phenomenon is the relapse of epileptic seizures and a brief duration of mitigation of seizure symptoms and/or seizure frequency in patients that are being treated with vagus nerve stimulation.

Poor response or non-responsiveness to stimulation may be due to a patient developing a tolerance to the effects of the stimulation. Such a tolerance could be achieved quite rapidly. For example, as discussed by Dr. Konstantinos Tsakalis in an as yet unpublished lecture, recent work at Arizona State University has suggested that electrical stimulation therapy applied in a rat model of epilepsy resulted in tolerance after approximately one month of treatment. The tolerance was indicated by an initial temporary reduction in seizure frequency followed by a relapse to baseline seizure frequency.

Additionally, it is not uncommon for an epileptic patient receiving VNS treatment to appear non-responsive at a medical checkup approximately 4-6 weeks after VNS implantation. In such a situation, the patient may have responded to the treatment for a short period of time (e.g., a few days, a week, or a couple of weeks) before tolerance was achieved, leading to the appearance of non-responsiveness at the checkup.

An analogy is the process of a bacteria strain adapting its genetic code to protect against a particular antibiotic. In order to kill a strain of bacteria that is immune to one antibiotic, another may have to be used. Thus, the present invention utilizes a random stimulation parameter adjustment to avoid the particular disorder or disorders being treated by the neurostimulation device from adapting to stimulations with identical parameters which are equally spaced in time, for example.

### BRIEF SUMMARY OF THE INVENTION

The present invention concerns electrical neurostimulation devices, and methods for using such devices. In a preferred embodiment of the invention, one or more stimulation parameters of each stimulation can be automatically and randomly adjusted such that any particular combination of stimulation parameters is not repeated for a given duration of time which could be as short as the subsequent stimulation or as long as it takes to exhaust all possible combinations of all adjustable neurostimulation parameters. This neurostimulation paradigm for treatment of a peripheral or central nervous system disorder or disorders (or other disorder(s)) may be used as the sole stimulation paradigm or may alternate between the aforementioned stimulation paradigm and a paradigm in which stimulations with identical parameters are equally spaced in time.

Any of a variety of neurostimulation devices can be modified to incorporate a random stimulation parameter adjustment and be utilized to make and use the invention. Examples of types of neurostimulation devices that may be used include, but are not limited to, vagus nerve stimulator (which is typically used in VNS therapy), and deep brain stimulation devices. Disorders of the central nervous system treated by one or more of the aforementioned stimulation devices include, but are not limited to, epilepsy, Parkinson's Disease, movement disorders, and depression.

In one embodiment of the devices and methods of the invention, the duration of the time interval between stimulations is randomly adjusted. Other parameters that may be altered individually or in any combination include, but are not limited to, stimulation frequency, current intensity, electrical pulse width, stimulation duration, and/or duration of time that a neurostimulation device is not stimulating. The random stimulation parameter adjustment can be achieved using devices and methods known in the art for random adjustment or modulation of an electrical signal.

The present invention provides electrical stimulation strategies and paradigms for commercial electrical stimulation apparatuses used for the treatment of disorders of the central nervous system, such as the vagus nerve stimulator used in VNS therapy.

The Vagus Nerve Stimulator, which is used in VNS therapy, is produced by Cyberonics Corporation. The present invention can increase the efficacy of neurostimulation treatment of nervous system disorders by providing a stimulation paradigm that is much less predictable than the paradigms currently in use. Therefore, the nervous disorders being treated by the neurostimulation device using the stimulation paradigm described herein are less likely to adapt to neurostimulation.

### DETAILED DESCRIPTION OF THE INVENTION

In one embodiment, the subject invention provides a method of neural stimulation that comprises applying a neural stimulation signal to at least one targeted nerve of a patient (e.g., to provide neurostimulation therapy); and automatically adjusting at least one parameter of the neural stimulation signal to avoid habituation to the neural stimulation signal. The patient may be suffering from one or more neurological disorders of the central nervous system and/or peripheral nervous system, or one or more other disorders. The patient may be a human or non-human (veterinary) patient or research animal.

Optionally, the method further comprises monitoring the patient for a desired or undesired response to the neural stimulation signal, and/or monitoring the patient for habituation to the neural stimulation signal.

Another aspect of the invention provides a neural stimulation device, comprising: a neural stimulation signal generator to generate an electrical signal (e.g., for neural stimulation therapy); and a modulator to automatically adjust at least one stimulation parameter of the neural stimulation signal. Optionally, a device further comprises a monitor for determining whether the patient is becoming habituated to the neural stimulation signal. Preferably, the monitor provides a signal to the modulator to adjust at least one stimulation parameter of the neural stimulation signal if the monitor detects that the patient is becoming habituated to the neural stimulation signal. Optionally, the monitor can be set such that predetermined physiological parameters indicative of adaptation will automatically trigger activation of the modulator. In one embodiment, the signal generator comprises one or more electrodes (also referred to herein as stimulation electrodes).

Any neural stimulation device known in the art can be modified to incorporate a random stimulation parameter adjustment and be utilized to make and use the invention. Examples of types of neural stimulation devices that may be used include, but are not limited to, a vagus nerve stimulator (which is typically used in VNS therapy), and deep brain stimulation devices.

In one embodiment, the device is a deep brain stimulation device, and the method of neural stimulation provides deep brain stimulation therapy.

Optionally, a neural stimulation device of the invention further comprises a tethered or on-board power source. Preferably, a neural stimulation device of the present invention comprises an onboard power source, such as a rechargeable or non-rechargeable battery. In some embodiments, the device possesses an internal battery capacity sufficient to allow a service life of greater than three years with the stimulus being a high duty cycle, e.g., virtually continuous, low frequency, low current stimulus pulses, or alternatively, the stimulus being higher frequency and amplitude stimulus pulses that are used only intermittently, e.g., a very low duty cycle. In one embodiment, the power source is an implant grade lithium ion battery as a primary battery.

Preferably, a neural stimulation device of the invention incorporates circuitry and/or programming to assure that the implantable signal generator will suspend stimulation, and perhaps fall-back to only very low rate telemetry, and eventually suspends all operations when the battery has discharged the majority of its capacity (i.e., only a safety margin charge remains). Once in this dormant mode, the implantable signal generator may provide limited communications and is in condition for replacement.

A neural stimulation device of the invention may be an external or implantable device. Thus, in some embodiments, a device can include components that operate outside the patient's body. The signal generator (e.g., stimulation electrode), and other components of the device (including the entire device) can be sized and configured to be implanted into a tissue region to apply a neural stimulation signal to at least one target nerve.

The neural stimulation may be applied with any type of electrical contact such as a lead placed in, on, around, or near any target nerve. The electrode or probe may be in contact with the target nerve, or it may be some distance (e.g., on the order of centimeters) away in cases in which it does not have to be in contact with the target nerve to activate it.

Stimulation may be applied through a lead, such as a fine wire electrode, inserted via needle introducer in proximity of a target nerve. When proper placement is confirmed, as indicated by patient sensation, visible movement of related organs, and/or by imaging device (such as magnetic resonance imaging (MRI), functional MRI (fMRI), PET scan, CT scan, etc.), the needle may be withdrawn, leaving the electrode in place.

Alternatively, stimulation may be applied through any type of nerve cuff (spiral, helical, cylindrical, book, flat interface nerve electrode (FINE), slowly closing FINE, etc.) that is surgically placed on or around a target nerve.

Stimulation may also be applied through a penetrating electrode, such as an electrode array that is comprised of any number (one or more) of needle-like electrodes that are inserted into a target nerve.

The lead may be routed subcutaneously to an implantable signal (e.g., pulse) generator. The signal generator may be located some distance from the electrode or it may be integrated with the electrode, eliminating the need to route the lead subcutaneously.

Control of the stimulation parameters may be provided by an external controller (also referred to herein as the modulator). The external controller may be a remote unit that uses wireless communication (such as RF or magnetic signals) to control the signal generator. The implantable signal generator may use regulated voltage (such as 10 mV to 20 V), regulated current (10 μA to 50 mA), and/or passive charge recovery to generate a stimulation waveform.

The stimulatory pulse may by monophasic or biphasic. In the case of the biphasic pulse, the pulse may be symmetrical or asymmetrical. Its shape may be rectangular or exponential or a combination of rectangular and exponential waveforms.

Pulses may be applied in continuous or intermittent trains (i.e., the stimulus frequency changes as a function of time). In the case of intermittent pulses, the on/off duty cycle of pulses may be symmetrical or asymmetrical, and the duty cycle may be regular and repeatable from one intermittent burst to the next or the duty cycle of each set of bursts may vary in a random (or pseudo random) fashion. As indicated above, varying the stimulus frequency and/or duty cycle may assist in limiting or preventing habituation because of the stimulus modulation.

In one embodiment, the stimulating frequency may range from 1 to 300 Hz. The frequency of stimulation may be constant or varying. In the case of applying stimulation with varying frequencies, the frequencies may vary in a consistent and repeatable pattern or in a random (or pseudo random) fashion or a combination of repeatable and random patterns.

A neural stimulation device of the invention may further comprise a housing or case to contain one or more components of the device. The housing can be implantable or remain external to the patient. The electronics within the device may be fabricated on a flexible or flex-rigid PC board using very high density techniques such as adhesive flip-chip or chip-on-board mounting, for example. The housing, signal generator, or other tissue contact materials used in the manufacture of the device are preferably biocompatible.

A neural stimulation device and method of the invention can incorporate wireless telemetry or inductively coupled telemetry for a variety of functions, such as those to be performed within arm's reach of the patient. Preferably, wireless telemetry is utilized. Such functions include receipt of programming and clinical parameters and settings from the clinician programmer, communicating usage history to the clinician programmer, and providing user control of the implantable signal generator. Each implantable signal generator may also have a unique signature that limits communication to only the dedicated controllers (e.g., the matched patient controller, or a clinician programmer configured for the implantable signal generator in question).

Any disorder or physiological dysfunction that is amenable to improvement by electrical stimulation of the nerves (by itself or with other therapies) may be treated using the device and method of the invention. Exemplified disorders include, but are not limited to, epilepsy, Parkinson's Disease, movement disorders, mood or behavior disorders, depression, chronic pain, and sexual dysfunction. The target nerve(s) will vary with the disorder(s) to be treated. Examples of neural stimulation devices that may be utilized in the device and method of the invention, disorders that can be treated using the device and method of the invention, and examples of corresponding target nerves and tissues, are described in U.S. Patent Publication Nos. 20070060954; 20070032837; 20070027500; 20070027499; 20070027498; 20070027496; 20070027492; 20070027484; 20070027483; 20060247721; 20060173507; 20060149337; 20060122659; 20060116737; 20060100671; 20060095090; 20060004422; 20060042183; 20060017749; 20060015153; 20050049650; 20050234523; 20050033372; 20050177192; 20050143789; 20050143787; 20050107842; 20050075680; 20040162590; 20040102828; 20040015205; 20040015204; 20040088009; 20030236558; 20030236557; 20030216792; and 20020193840; which are each incorporated by reference herein in their entirety.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method of neural stimulation, comprising:
applying a neural stimulation signal to at least one targeted nerve of a patient; and
automatically adjusting at least one parameter of the neural stimulation signal to avoid habituation by the patient to the neural stimulation signal.

2. The method according to claim 1, further comprising monitoring the patient for a desired or undesired response to the neural stimulation signal.

3. The method according to claim 1, further comprising monitoring the patient for habituation to the neural stimulation signal.

4. The method according to claim 3, wherein the at least one parameter of the neural stimulation signal is automatically adjusted if the patient is habituating to the neural stimulation signal.

5. The method according to claim 1, wherein applying a neural stimulation signal to at least one targeted nerve of a patient comprises using an electrode placed on or near the at least one targeted nerve.

6. The method according to claim 1, wherein applying a neural stimulation signal to at least one targeted nerve of a patient comprises using a nerve cuff placed on or around the at least one targeted nerve.

7. The method according to claim 1, wherein a frequency of the neural stimulation signal is from 1 Hertz to 300 Hertz.

8. A neural stimulation device, comprising:
a neural stimulation signal generator for generating a neural stimulation signal; and
a modulator for automatically adjusting at least one parameter of the neural stimulation signal.

9. The neural stimulation device according to claim 8, further comprising a monitor for monitoring whether a patient is becoming habituated to the neural stimulation signal.

10. The neural stimulation device according to claim 9, wherein the monitor provides a signal to the modulator to adjust at least one parameter of the neural stimulation signal if the monitor detects that the patient has become habituated to the neural stimulation signal.

11. The neural stimulation device according to claim 8, further comprising an on-board power source.

12. The neural stimulation device according to claim 11, wherein the on-board power source comprises an implant grade lithium ion battery.

13. The neural stimulation device according to claim 12, further comprising circuitry, wherein the circuitry causes the neural stimulation signal generator to stop generating a neural stimulation signal when the implant grade lithium ion battery has discharged its capacity except for a safety margin charge.

14. The neural stimulation device according to claim 8, wherein, in use, the neural stimulation signal generator is implanted into a patient's body.

15. The neural stimulation device according to claim 8, wherein the modulator communicates with the neural stimulation signal generator using wireless communication.

16. The neural stimulation device according to claim 8, further comprising a housing.

17. The neural stimulation device according to claim 8, wherein the neural stimulation device utilizes wireless telemetry.

18. The neural stimulation device according to claim 8, wherein a frequency of the neural stimulation signal is from 1 Hertz and 300 Hertz.

19. The neural stimulation device according to claim 8, wherein the neural simulation device is a vagus nerve stimulator.

20. The neural stimulation device according to claim 8, wherein the neural stimulation device is a deep brain stimulation device.
